(19) United States
(12) Patent Application Publication
(10) Pub. No.: US 2012/0148513 A1
(43) Pub. Date: Jun. 14, 2012

(54) SUNSCREEN-CONTAINING DISSOLVABLE FILMS

(75) Inventors: Robert B. Fletcher, Midland, MI (US); Wei H. Yu, Edison, NJ (US); Tatiana V. Drovetskaya, Basking Ridge, NJ (US)

(73) Assignee: DOW GLOBAL TECHNOLOGIES INC., Midland, MI (US)

(21) Appl. No.: 12/825,832

(22) Filed: Jun. 29, 2010

Related U.S. Application Data
(60) Provisional application No. 61/221,878, filed on Jun. 30, 2009.

(51) Int. Cl. A61K 8/73; A61Q 17/04

(52) U.S. Cl. 424/60; 424/59

(57) ABSTRACT

The present invention provides personal care dissolvable films, comprising water soluble cellulose ether and a sunscreen agent.

### CROSS-REFERENCE TO RELATED APPLICATION(S)

This application claims benefit of priority from U.S. Provisional Patent Application No. 61/221,878, filed Jun. 30, 2009, which application is incorporated by reference herein in its entirety.

### FIELD

The present invention relates to sunscreen-containing dissolvable films.

### BACKGROUND

Dissolvable films are an exciting development in the personal care industry, in theory, offering unparalleled portability. Compact and lightweight, the film can be wetted with an aqueous liquid to form a wet product that can then be applied to a user when desired.

Historically, dissolvable films struggle with aesthetic properties. For example, they can be tacky, as a result of a combination of polymers and plasticizers present. Similarly, some films do not dissolve fast enough and therefore can feel grainy, lumpy, or stringy as a result of relatively long disintegration times. The success of a personal care product depends in great measure on the way it appears and feels to a user at the time of use. For example, it is not intuitive to create a leave-on product from tack-causing ingredients. Merely removing the tack-causing ingredients is not an option, as it may negatively impact a film's mechanical properties. Similarly, lowering disintegration times can result in a wet product that runs through the fingers instead of being appropriately viscous and may actually increase tackiness.

Moreover, inorganic sunscreens themselves can be challenging to formulate. Many countries now require quantifiable UVA protection on sunscreen labels, and inorganic sunscreens like zinc oxide and titanium dioxide are two of the few filters that offer UVA protection. However, high concentrations of inorganic sunscreens can cause an undesirable white appearance when applied to the skin. Then too, a dissolvable film can only incorporate so much active ingredient before losing desirable properties.

Thus, what is needed are sunscreen-containing dissolvable films.

### SUMMARY

In one embodiment, the present invention provides personal care dissolvable films, comprising a water soluble cellulose ether and a sunscreen agent.

### DETAILED DESCRIPTION

In one embodiment, the present invention provides personal care dissolvable films, comprising a water soluble cellulose ether and a sunscreen agent. Water soluble cellulose ether includes methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.

Preferably, the water soluble cellulose ether is hydroxypropyl methylcellulose. Hydroxypropyl methylcellulose is generally available under the tradename METHOCEL E, F, J, and K (The Dow Chemical Company). The polymeric backbone of cellulose is a repeating structure of anhydroglucose units. Treatment of cellulosic fibers with caustic solution and methyl chloride and propylene oxide, yields cellulose ethers substituted with methoxy groups and hydroxypropyl groups. The term “DS” refers to the degree of methoxyl substitution per anhydroglucose unit. The term “MS” refers to the degree of hydroxypropoxyl substitution per anhydroglucose unit. Each grade is differentiated by the methoxy and hydroxypropyl substitution on the polymeric backbone. Hydroxypropyl methylcellulose useful in the present invention has a DS of less than 2.0, preferably, the DS is about 1.8 or about 1.4, and an MS in a range of about 0.05 to about 1.2, preferably about 0.1 to about 0.5 More preferably, the water soluble cellulose ether is hydroxypropyl methylcellulose with a DS of 1 to 2.

A film forming composition is mixed, and this composition is cast and dried to create a dissolvable film. In one embodiment of the present personal care dissolvable film, the water soluble cellulose ether is present in an amount from about 1% to about 50% by weight of the dry film, preferably about 10 to about 35%.

“Weight percent” refers to the weight of the component in a theoretical completely dried film, in other words, as if the film had been dried until only nonvolatile components remained. Thus, for this application, weight percent is independent of humidity.

In one embodiment, the sunscreen agent is at least one of octyl methoxycinnamate, avobenzone, para aminobenzoic acid, homosalate, titanium dioxide, zinc oxide, benzophenones, benzylidenes, salicylates, or other known UV filters.

In one embodiment, the sunscreen agent is an inorganic sunscreen selected from zinc oxide (ZnO), titanium dioxide (TiO₂), or mixtures thereof. In one embodiment, the inorganic sunscreens are pigment grade zinc oxide or pigment grade titanium dioxide. Most pigment grade particles used in a sunscreen produce a cosmetically undesirable white appearance caused by light scattering. Thus, in one embodiment, the inorganic sunscreens are transparent zinc oxide or transparent titanium dioxide. Thus, the term “transparent,” as used herein has a special meaning, referring to inorganic metal oxide sunscreen particles produced by a variety of processing conditions which render the inorganic metal oxide compositions as clear, or transparent, upon application. In other words, these specially processed inorganic metal oxide compositions do not appear white once applied, hence the moniker of transparent.

Examples of transparent zinc oxide are disclosed in, for example, U.S. Pat. Nos. 5,223,250, 5,372,805, 5,573,753, 5,587,148, and 5,876,688. One example of a transparent zinc oxide is commercially available under the tradename Z-COTE from BASF Corporation (Germany). Another example of transparent zinc oxide is commercially available under the tradename ZINCLEAR IM from Antaria Limited (Australia). Another example of transparent zinc oxide is commercially available under the tradename Z-CLEAR from Actifirm (USA).

Examples of transparent titanium dioxide are disclosed in, for example, U.S. Pat. Nos. 5,573,753, 5,733,895, and 7,390,355. Examples of transparent titanium dioxide are commercially available under the tradenames TIPAQUE and TTO-51(A) from Ishihara Sangyo Kaisha, Ltd. (Japan). Another example of a transparent titanium dioxide is commercially available under the tradename T-COTE from BASF Corporation (Germany). Another example of transparent titanium dioxide is commercially available under the tradename UFTR from Miyoshi Kasei (Japan). Another example of transparent titanium dioxide is commercially available under the tradename SOLAVEIL CLARUS from Uniqema (Great Britain).

In one embodiment, the transparent zinc oxide is present in an amount from about 1% to about 50% by weight of the dry film, preferably about 2 to about 10%.

In another embodiment, the sunscreen agent is an organic sunscreen.

In yet another embodiment, the sunscreen agent is a mixture of inorganic and organic sunscreens. Preferably, zinc oxide or titanium dioxide and octyl methoxycinnamate (OMC).

In one embodiment, the personal care dissolvable film contains less than 0.5% of modified starch.

Composition of the present invention can further incorporate other ingredients known in the art of sun care formulations and dissolvable films. Other optional ingredients for personal care compositions of the present invention include at least one of the following: additional film forming agents, cosmetically acceptable emollients, moisturizers, conditioners, oils, sunscreens, surfactants, emulsifiers, preservatives, rheology modifiers, colorants, preservatives, pH adjustors, propellants, reducing agents, fragrances, foaming or de-foaming agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides.

The moisturizers include 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, and polyols, such as, for example, propylene glycol, glycerol and sorbitol.

Conditioners include stearalkonium chloride, dicetyldimonium chloride, lauryl methyl gluceth-10 hydroxypropyldimonium chloride, and natural and synthetic conditioning polymers such as polyquaternium-4, polyquaternium-7, polyquaternium-10, polyquaternium-24, polyquaternium-67 and the like, chitosan and derivatives thereof.

Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID 350 silicone fluid (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes from the list above may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances may be aldehydes, ketones, or oils obtained by extraction of natural substances or synthetically produced as described above. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

### EXAMPLES

The following examples are for illustrative purposes only and are not intended to limit the scope of the present invention. All percentages are by weight unless otherwise specified.

### Example 1

Exemplary personal care dissolvable film-forming compositions contain the components recited in TABLE 1.

TABLE 1

| | | | Batch | Batch |
|---|---|---|---|---|
| | | Batch 1 | 2 | 3 |
| A | Propylene Glycol | 8.0 | 4.0 | 8.0 |
| | HPMC-DS~1.9; MS~0.23 | 15.0 | 14.0 | — |
| | HPMC-DS~1.4; MS~0.21 | — | — | 15.0 |
| | Water | 56.46 | 62.16 | 56.46 |
| B | SEPIGEL 305 Polyacrylamide/C13-14 | 1.0 | 0.8 | 1.0 |
| | Isoparaffin/Laureth-7 | | | |
| | DC 193 dimethicone copolyol | 3.5 | 3.0 | 3.5 |
| C | ZINCLEAR IM_AB (50% active) | 9.0 | — | 9.0 |
| | transparent zinc in C₁₂₋₁₅ Alkyl Benzoate | | | |
| | KOBO INH70T6 (67% active) Titanium | — | 9.0 | — |
| | Dioxide in isononyl isononanoate, | | | |
| | polyglycerol-6 polyricinoleate, and | | | |
| | isoprpropyl titanium triisostearate | | | |
| | JEESCREEN OMC Ethylhexyl | 3.0 | 5.0 | 3.0 |
| | Methoxycinnamate | | | |
| | DERMOBLOCK OS 2-Ethylhexyl salicylate | 2.0 | — | 2.0 |
| D | DC 9509 Dimethicone/Vinyl Dimethicone | 2.0 | 2.0 | 2.0 |
| | Crosspolymer (and) C12-14 Pareth-12 | | | |
| E | NIPAGUARD Propylene Glycol, | 0.04 | 0.04 | 0.04 |
| | Diazolidinyl Urea, Methyl Paraben, Propyl | | | |
| | Paraben | | | |

Part A ingredients are combined and mixed at temperature ˜90° C. until the HPMC completely hydrates. Then the Part A is cooled down to room temperature and mixed until the polymer completely dissolves. Part B and C ingredients are added to Part A and mixed until the dispersion is uniform. The rest of ingredients are added to the batch in order and mixed until uniform. The films were cast by placing a few grams of material along the inside of a 15 mil die at the top of a release sheet held into place on a Leneta draw down vacuum plate while pulling a vacuum. The formulation is drawn down the release sheet using the 15 mil die to make a smooth, uniform film of about 3.2 mil. The film is dried in a 90° C. oven for 15 min and then stored at constant humidity and temperature.

### Example 2

### Comparative

Exemplary dissolvable film-forming compositions contain the components recited in TABLE 2.

TABLE 2

| | | Comparative |
|---|---|---|
| | | Batch A |
| A | Propylene Glycol | 8.0 |
| | Pullulan | 15.0 |
| | Water | 56.46 |
| B | SEPIGEL 305 Polyacrylamide/C13-14 Isoparaffin/ | 1.0 |
| | Laureth-7 DC 193 dimethicone copolyol | 3.5 |
| C | ZINCLEAR IM_AB (50% active) transparent zinc | 9.0 |
| | in C₁₂₋₁₅ Alkyl Benzoate | |
| | JEESCREEN OMC Ethylhexyl Methoxycinnamate | 3.0 |
| | DERMOBLOCK OS 2-Ethylhexyl salicylate | 2.0 |
| D | DC 9509 Dimethicone/Vinyl Dimethicone | 2.0 |
| | Crosspolymer (and) C12-14 Pareth-12 | |
| E | NIPAGUARD Propylene Glycol, Diazolidinyl Urea, | 0.04 |
| | Methyl Paraben, Propyl Paraben | |

Mixing and film forming procedures are substantially as described in Example 1.

### Example 3

Sunscreen compositions prepared substantially according to the protocols of Examples 1 and 2 were prepared and cast to create films. The films were dissolved as follows: A 2 cm×3 cm piece of dissolvable film is placed in the palm of the operator's left hand. 2 ml of water are added and the operator rubs the film with the water using two fingers of the right hand in a circular motion (each circle taking approximately one second) until the film is completely dissolved. Dissolution times under 15 seconds are considered acceptable. The SPF values of the dissolved films are determined and recited in TABLE 3.

TABLE 3

| | | | | Comparative |
|---|---|---|---|---|
| | Batch 1 | Batch 2 | Batch 3 | Batch A |
| SPF | 35 | 47* | 45 | 5.4 |

*Batch 2 was measured at a separate time and place, but according to substantially the same protocol.

The SPF was determined using an in vitro technique substantially according to the following protocol:

A piece of TRANSPORE tape, supplied from 3M, is cut from the roll, and the surface area is measured. The dissolvable film for SPF measurement is then weighed. The dissolvable film is then placed on top of the tape and a known weight (about 0.9 g) of water is added drop wise to the film, to form a lotion. The lotion is then spread evenly on the tape using a finger cot. The concentration of the film on the tape is calculated by:

$$Film Conc . ( mg / {cm}^{2} ) = \frac{weight of diss . film + weight of water}{Surface area of transpore tape}$$

The lotion is allowed to dry. The UV absorption of the dry uniform layer is measured using a LABSPHERE 1000S spectrometer at multiple points on the layer, using this information, the SPF can be calculated by the following equation:

$$S P F = \frac{{\int }_{290 nm}^{400 nm} E ( λ ) S ( λ ) \partial λ}{{\int }_{290 nm}^{400 nm} E ( λ ) S ( λ ) {10}^{( - A ( λ ) )} \partial λ}$$

Where E(λ)=spectral irradiance of the Standard Sun Spectrum; S(λ)=erythemal action spectrum at wavelength λ; and A(λ)=spectral absorbance at wavelength λ.

The SPF that is measured above is then converted to a calculated SPF to extrapolate to 2 mg/cm² of lotion coverage on the tape using the following equation:

$$Calculated S P F = Measured S P F \times \frac{2 mg / cm 2}{actual film concentration}$$

The results in Table 3 are surprising, indicating a synergistic effect of some type.

### Example 4

As aesthetic properties are of paramount importance in dissolvable films, the aesthetic properties of a high SPF film inventive film (dried Batch 2) were tested against a traditional sunscreen lotion (BLUE LIZZARD SPF 30 for sensitive skin lotion—Hereinafter Comparative Batch B).

In-situ formulation was prepared by dissolving 1.6 g of dry film (dried Batch 2) in 12 g of water and stiffing until complete dissolution was achieved. Then, 10 expert panelists trained to evaluate performance of products received as 0.05 g of the in situ Batch 2 lotion in a 1.5″×2.5″ area of one forearm, and 0.05 g of a benchmark of Comparative Batch B lotion to the other forearm for comparison. Panelists spread the sample with index and middle fingers in a circular motion until the sample is evenly distributed. The panelists were asked to pick one product that has better performance parameters shown in the Table 4.

TABLE 4

| | Ease of | Speed of | Tacki- | | White- | Overall |
|---|---|---|---|---|---|---|
| | Spread | Absorption | ness | Slip | ness | Skin Feel |
| Batch 2 | 8 | 7 | 4 | 8 | 4 | 8 |
| Compara- | 2 | 3 | 6 | 2 | 6 | 2 |
| tive | | | | | | |
| Batch B | | | | | | |

The score from the evaluation represents number of preference by the panelists in the test. The result shows that the inventive film is easier to be spread on the skin, has quicker absorption, better slippery skin feel, and much better overall skin feel at initial application.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A personal care dissolvable film, comprising:
a water soluble cellulose ether; and
a sunscreen agent.

2. The personal care dissolvable film of claim 1, wherein the water soluble cellulose ether is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.

3. The personal care dissolvable film of claim 1, wherein the water soluble cellulose ether is hydroxypropyl methylcellulose.

4. The personal care dissolvable film of claim 1, wherein the water soluble cellulose ether is present in an amount from about 1% to about 50% by weight of the dry film.

5. The personal care dissolvable film of claim 1, wherein the sunscreen agent is at least one of octyl methoxycinnamate, avobenzone, para aminobenzoic acid, homosalate, titanium dioxide, or zinc oxide.

6. The personal care dissolvable film of claim 1, wherein the sunscreen agent is an inorganic sunscreen selected from zinc oxide, titanium dioxide, or mixtures thereof.

7. The personal care dissolvable film of claim 6, wherein the sunscreen agent is selected from transparent zinc oxide or transparent titanium dioxide.

8. The personal care dissolvable film of claim 1, wherein the sunscreen agent is an organic sunscreen.

9. The personal care dissolvable film of claim 1, further comprising at least one emollient, plasticizer, detackifying agent, emulsifier, colorant, fragrance, or preservative.

10. The personal care dissolvable film of claim 1, wherein the film contains less than 0.5% modified starch.

11. A sunscreen lotion, comprising:
the personal care dissolvable film of claim 1; and
an aqueous component present in an amount sufficient to dissolve the film.
